(19) United States
(12) Patent Application Publication
(10) Pub. No.: US 2014/0316251 A1
(43) Pub. Date: Oct. 23, 2014

(54) MRI METHOD FOR INVESTIGATING INTERSTITIAL FLUID VELOCITY IN A TISSUE USING A NULLING PREPARATION

(71) Applicant: Simon Walker-Samuel, Reigate Surrey (GB)

(72) Inventor: Simon Walker-Samuel, Reigate Surrey (GB)

(73) Assignee: UCL BUSINESS PLC, London (GB)

(21) Appl. No.: 14/343,716

(22) PCT Filed: Sep. 6, 2012
(86) PCT No.: PCT/GB2012/052193

§ 371 Date: Jun. 23, 2014

(30) Foreign Application Priority Data
Sep. 7, 2011 (GB) 1115452.3

(51) Int. Cl. G01R 33/56; G01R 33/563; A61B 5/00; G01R 33/54; A61B 5/026; G01R 33/48

(52) U.S. Cl. CPC G01R 33/5601; A61B 5/0263; G01R 33/4828; A61B 5/742; G01R 33/546; A61B 5/7275; G01R 33/5635
USPC 600/419

(57) ABSTRACT

A magnetic resonance imaging (MRI) method of investigating tissue fluid velocity in a region of interest comprising substantially eliminating the magnetic resonance signal of blood flowing in one or more blood vessels in the tissue by applying a nulling preparation to the tissue; and deriving the interstitial fluid velocity using magnetic field gradients.

The present invention relates to magnetic resonance imaging (MRI). MRI uses a large, static magnetic field to align magnetic moments of protons in living tissue. By applying radio frequency (RF) radiation at the resonance frequency of these protons, their magnetisation can be reoriented by a pre-specified angle, which induces a signal in a receiver coil. The detected signal derives mainly from water and fat protons due to their natural abundance in the human body.

The present invention relates more specifically to study of interstitial fluid velocity and fluid pressure within tissue using MRI techniques. One tissue of particular interest is tumour tissue. Due to their high vascular permeability and poor vascular function, tumours exhibit raised interstitial fluid pressure (IFP).

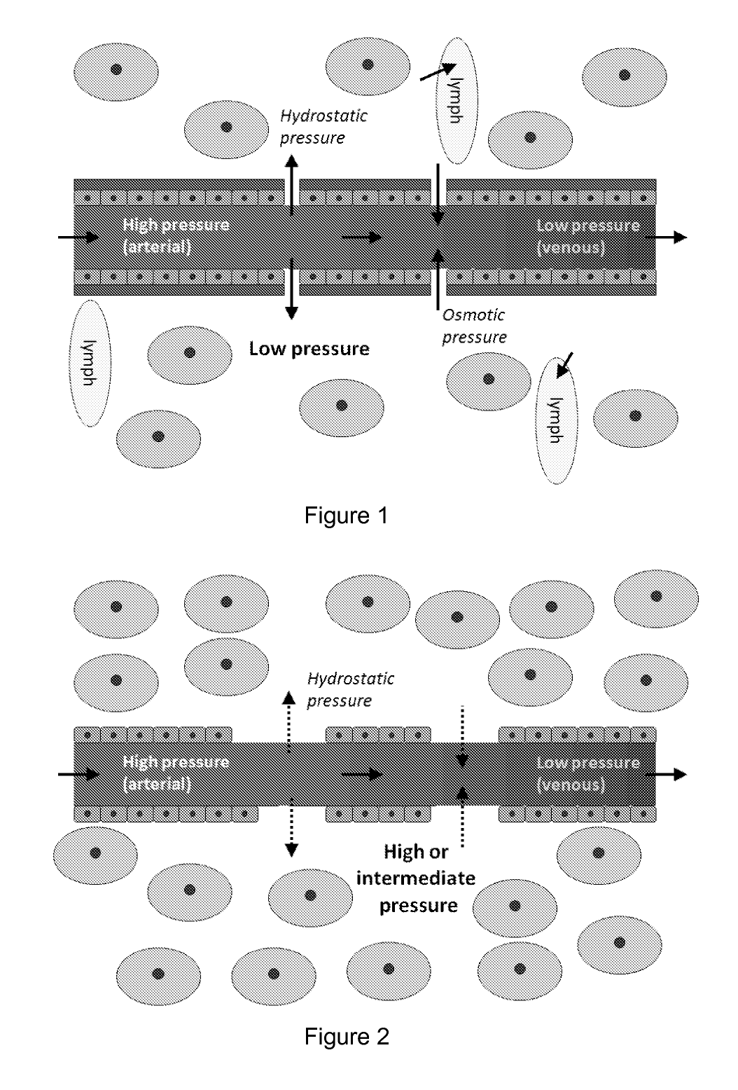

FIG. 1 shows fluid exchange in normal tissues. Exchange is moderated by hydrostatic and osmotic pressure gradients between blood vessels and tissue interstitium. Tissue fluid can also be extracted via lymph vessels.

FIG. 2 shows fluid exchange in tumours. High vascular permeability results in excessive fluid leakage from blood vessels into the interstitium and the removal of osmotic pressure gradients. This can impact significantly on the delivery of drugs, for example anti-cancer drugs.

Interstitial fluid pressure has been shown in tumour xenograft models to be heterogeneously distributed, with high pressure in the centre and lower pressure towards the periphery [1]. This raised pressure impacts on drug delivery [2], is implicated in the development of metastasis [3] and induces a radial convection of fluid through the interstitium.

For the above reasons, interstitial fluid velocity (IFV) is of interest in the study of tumour tissue and other tissue. However it has not yet been reliably investigated due to the lack of non-invasive and reliable technologies.

The gold standard for measurement of interstitial fluid pressure (IFP) is by inserting a needle containing a pressure sensor. This is known as the ‘wick-in-needle’ technique. This procedure is highly invasive and the insertion of the probe can perturb the fluid pressure. As raised IFP is a ubiquitous marker of disease, non-invasive methods for its measurement in the clinic would be extremely advantageous. Moreover, the potential to assess response to treatment (it would be anticipated that a lowering of IFP would be associated with a positive response to therapy) or for evaluating strategies for lowering IFP in order to improve drug delivery, would most likely be of interest to pharmaceutical companies. Furthermore, such a technique may be of use in the brain, for example, to assess damage caused by stroke.

In 2006 Hassid et al. [7] proposed a method using MRI for measuring tumour IFP, but which has not found wide utility. This was because it required a long infusion of contrast agent (up to two hours), had to assume a fixed value for the size of the extra-vascular, extra-cellular volume, required a calibration for converting MRI data to absolute IFP (which were themselves derived from wick-in-needle measurements) and could not distinguish between the delivery of contrast agent by the vasculature and by convection currents within the interstitium.

Thus it would be desirable to provide a way of investigating fluid flow and/or pressure within tissue that overcomes or mitigates at least some of the disadvantages of the prior art. Although such a method would have one application in the investigation of tumours, parallel applications present themselves in any living tissue which could benefit from particular investigation. For example, stroke tissue or tissue affected by compartment syndrome.

### Statements of Invention

The invention is defined in the independent claims, to which reference should now be made. Advantageous embodiments are set out in the sub claims.

According to an embodiment of a first aspect there is provided a magnetic resonance imaging (MRI) method of investigating fluid flow in a region of interest of a tissue comprising: substantially eliminating the magnetic resonance signal of blood flowing in one or more blood vessels in the tissue by applying a nulling preparation to the tissue; and deriving the interstitial fluid velocity using magnetic field gradients.

Standard MRI sequences cannot be used to probe extra vascular currents because these are obscured by blood flowing within the vasculature. Thus the slower fluid flow within the tissue cannot easily be detected with standard MRI conditions. The inventor has come to the realisation not only that would it be advantageous to study fluid flow associated with interstitial fluid pressure but also that the fluid flow can be investigated by removing influence of the blood flowing more quickly in blood vessels in the tissue by using a nulling preparation. The nulling preparation can include a sequence of pulses. Subsequently, velocity of the interstitial fluid can be determined using an MRI technique.

In preferred embodiments, the nulling preparation includes an inversion pulse applied to the tissue by an MRI apparatus to change the polarity of magnetism induced by a static field of the MRI apparatus. An inversion pulse is intended to change the polarity of magnetism induced by the static field of the MRI apparatus. In many preferred embodiments it has the effect of inverting the magnetism of the blood with respect to the “equilibrium” polarity included by the static field, but it may also have a different angular effect. It is thus also contemplated to use any inversion pulse that changes the polarity of magnetism to an extent that allows the magnetism of the blood to be reoriented. As an aside, reference to the magnetism polarity/direction of the blood is of course to the magnetism direction of the water protons within the blood.

Preferably, the inversion pulse is global, that is not spatially limited to any particular part of the tissue for investigation. Thus the inversion pulse may be applied to the whole of the subject which is situated within the MRI apparatus.

In some embodiments, differentiation between the blood flowing in the blood vessels and from the fluid flow within the tissue can be achieved simply by measuring velocity of fluid flow at a time when any potential resonance signal from the blood is nulled (that is, the blood has no useable magnetisation). The inversion pulse changes the magnetisation of the tissue and the blood, which immediately begin to recover back towards equilibrium at an exponential rate. This rate of recovery can differ between blood and the surrounding tissue and thus velocity measurement can be taken at a time when the signal from the blood is nulled and signal from the tissue is not.

In preferred embodiments, the nulling of the blood is achieved by applying a 180° RF pulse to completely invert the magnetisation of the tissue.

Preferably, a further inversion pulse is then applied to a region of interest of the tissue, to reorient magnetism of the tissue in the region of interest with the original static field magnetism.

This additional pulse (which may be a second 180° pulse RF following the first 180° pulse) has the localised effect of instantly realigning only the region of interest to equilibrium, thereby allowing the full equilibrium magnetisation of the tissue in the region of interest to be utilised for signal formation.

The region of interest may be any region suitable for investigation and is preferably an MRI slice of an appropriate thickness. The thickness of the second pulse may be between about 0.3 and 4 mm, preferably between about 0.5 and 3 mm. Suitable range of thicknesses for pre-clinical work might be 0.5 to 1 mm and for clinical work 2 to 3 mm.

Preferably, velocity is derived in the region of interest after a flow time sufficient to allow blood in the region of interest to be substantially replaced by inflowing blood from outside the region of interest. Thus any blood flowing into the region of interest will not have experienced any second or rectifying pulse and will still be recovering from the first pulse. The flow time required may be very short, perhaps in the order of 1s or less, especially if the region of interest is a thin slice (of a few millimetres thickness) and the blood vessel is perpendicular to the imaging slice.

In preferred embodiments, a velocity encoding sequence is applied to derive the interstitial fluid velocity in the region of interest. Thus velocity encoding is used to measure the convection of fluid through tissue. Such velocity encoding sequences have been used previously to measure blood velocity in major arteries [9]. However there has been no previous use of velocity encoding for investigation of fluid flow in tissues.

The optimal time for the velocity encoding sequence or other velocity measurement to be applied is (after the flow time) and at least approximately at a recovery time t_{rec}=log(2)T_{1, blood}, where T_{1, blood} is the T₁ spin-lattice relaxation time of blood and t_{rec} is the time taken for the blood to recover from full inversion to the null point. At this time the blood would have recovered half way between maximum inversion and equilibrium magnetisation and as such have no usable magnetisation. At this point, no signal can be acquired from it and the blood is effectively nulled (without any net longitudinal magnetisation). By acquiring an image at this point using standard phase and frequency encoding techniques, only protons within the tissue provide a signal.

The velocity encoding sequence may comprise in each of one or more directions, two applications of a sequence, the second using gradients of a different polarity, and the difference in phase between echo measurements (measurements of emission/magnetic resonance signals emitted by the tissue) after the two applications being proportional to the velocity. Here, magnetic field gradients may be used to provide a linear variation of a strength of a field across the sample/tissue. Fluid flowing within the interstitium experiences a range of field strengths depending on the direction and magnitude of the motion, and therefore, while the gradient is applied will accumulate a phase offset.

Preferably the encoding sequence is applied in one, two or preferably three orthogonal spatial orientations. Advantageously, by applying field gradients in three orthogonal directions and using multiple gradient strengths, a changing phase of the acquired signal caused by each gradient setting can be used to measure interstitial fluid velocity (IVF).

The velocity can then be calculated, preferably by taking into account the gyromagnetic ratio of water protons and the difference in first order velocity encoding gradient moments for the two measurements.

The velocity encoding sequence can be applied consecutively on numerous slices, to build up a 3-dimensional image or using a three-dimensional, slab-selective acquisition with two phase encoding directions. Furthermore, acceleration strategies such as echo planar imaging (EPI) could be used to decrease the acquisition time.

Once the velocity of the fluid in the tissue has been derived, it may be displayed, for example on a screen, printout or in any other fashion.

Returning to the beginning of the method, in order to find the recovery time accurately, T₁ of blood may be measured as a first process. For example, the T₁ of vascular blood in the heart may be measured. Alternatively, the relaxation time TI may be found from tables and approximations as known in the state of the art.

The tissue investigation of the present invention may be used to predict drug deficiency and/or response to therapy of the tissue. The tissue may be tumour tissue, stroke-damaged tissue, tissue affected by a compartment syndrome or any other tissue for investigation.

According to an embodiment of a further aspect there is provided a method of measuring pressure of tumour interstitium comprising using the tissue investigation method according to any of the variants described hereinbefore and then using interstitial fluid velocity to calculate interstitial fluid pressure.

The Navier-Stokes equation can be applied and adapted in this context. In one preferred embodiment, numerical calculus of the velocity vector field can provide a first-order estimate of pressure gradients.

Preferably, the pressure is calculated by applying diffusion MRI sequences to investigate diffusion within the tissue in conjunction with use of the Navier-Stokes and Einstein-Stokes equations to derive pressure. This allows a more accurate picture to be formed, by taking viscosity into account.

For example, in one diffusion MR technique, a spin-echo sequence is used for image acquisition with the addition of linear field gradients, for example at equal time separation from the refocusing radio frequency pulse. Such pulses are of sufficient magnitude and separation to be sensitive to the diffusion of water within the tissue. This procedure is analogous to the velocity encoding procedure above, but utilises the change in signal magnitude induced by the gradients, as opposed to the change in phase. As such it is sensitive to both coherent and incoherent flow and the spacing and amplitude of the gradients can be manipulated to provide quantification of water diffusion at specific length scales, for example via a parameter known as the b-value. By modelling the decrease in water signal as an exponential decay with increasing b-value, the apparent diffusion coefficient (ADC) of water can be estimated. Using b-values of greater than 250 mm²/s the coherent aspect of the signal is diminished and purely incoherent, diffusive transport is measured. The ADC can then be converted to an apparent viscosity measure using the Einstein-Stokes equation, which is utilised in the Navier-Stokes equation, along with IFV measurements, to estimate IFP spatial gradients. Thus, incoherent, diffusive transport acts as a local probe of the hydraulic conductivity of the surrounding medium, whilst the coherent, convective transport defines the directionality and magnitude of flow gradients.

According to an embodiment of a further aspect of the invention there is provided a magnetic resonance imaging (MRI) apparatus for investigating interstitial fluid velocity in a tissue of a subject, the MRI apparatus including a controller operable to control the applied magnetic fields within the MRI apparatus to: substantially eliminate magnetic resonance of blood flowing in one or more blood vessels in the tissue by application of a nulling sequence; and apply a velocity encoding sequence allowing derivation of the interstitial fluid velocity.

Such an MRI apparatus suitably includes conventional hardware in terms of coils and control electronics and the defined controller (sometimes referred to as a CPU) to control the applied magnetic fields in accordance with the invention.

According to an embodiment of another aspect there is provided a computer which is programmed to accept data from the magnetic resonance imaging (MRI) apparatus described hereinbefore, wherein the data includes echo data representing signals emitted from the tissue in response to the velocity encoding sequence and the computer is also programmed to derive interstitial fluid velocity by processing the echo. The skilled reader will appreciate that a computer may be provided as part of the MRI apparatus (for example as a console link to the CPU with appropriate input and output facilities for the user) or the computer may be provided separately, for example remotely from the MRI apparatus.

In either variant, the computing functionality may further comprise a user interface including an output device to allow the user to view an image of interstitial fluid velocity and an input device to allow the user to control the MRI apparatus programming.

The combination of the MRI apparatus defined hereinbefore and computer defined hereinbefore may be referred to as an MRI system.

According to an embodiment of a further aspect there is provided a computer program which when executed by an MRI system causes it to carry out the method of any of the variants described hereinabove. The invention also extends to a tangible non-transitory computer-readable medium storing such a computer program. A computer program embodying the invention may be stored on such a computer-readable medium, or it could, for example, be in the form of a signal such as a downloadable data signal provided from an Internet website, or it could be in any other form.

In any of the above aspects, the various features may be implemented in hardware, or as software modules running on one or more processors.

The skilled reader will appreciate that the features and the preferable features of any or all of the different aspects and embodiments may be combined. Thus for example the controller of the MRI apparatus may control the MRI apparatus to carry out any of the preferable method steps of the method aspect.

For a better understanding of the present invention, and to show how the same may be carried into effect, reference will now be made, by way of example, to the accompanying drawings, in which:

FIG. 1 shows fluid exchange in normal tissues;

FIG. 2 shows fluid exchange in tumours;

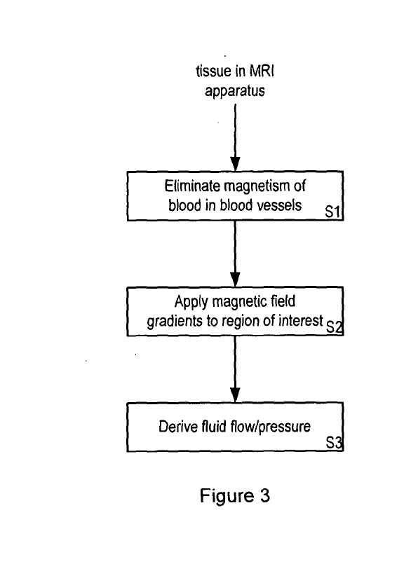

FIG. 3 shows a general embodiment of the invention;

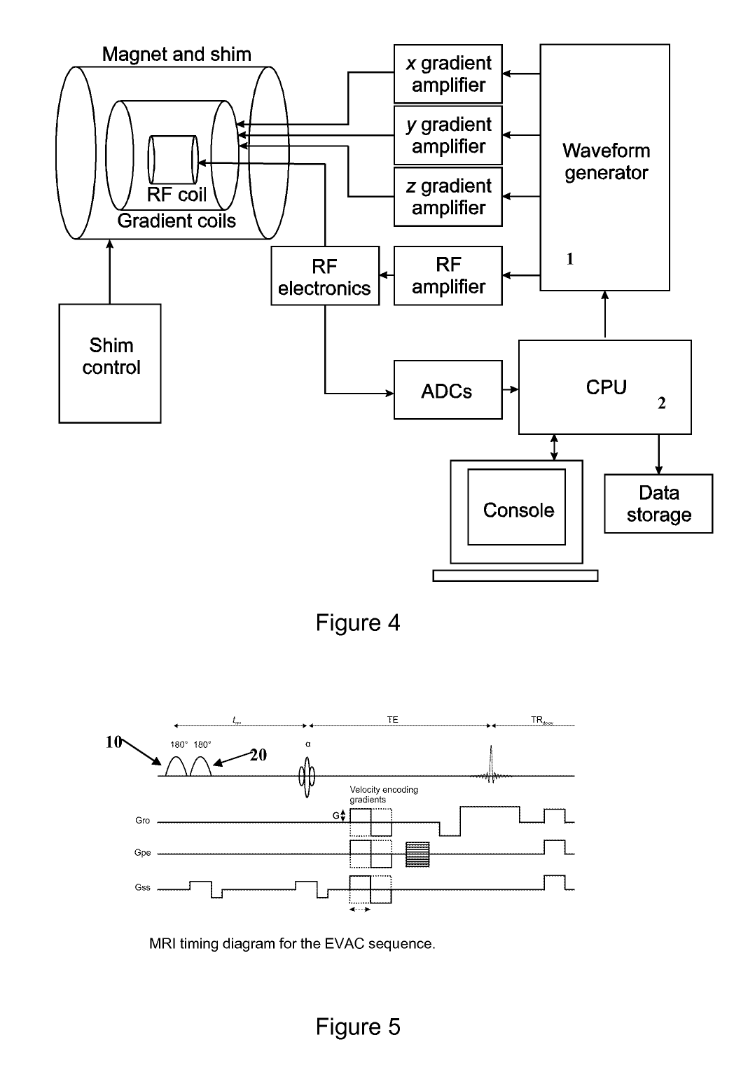

FIG. 4 shows a schematic diagram of a magnetic resonance imaging apparatus;

FIG. 5 shows an MRI timing diagram for the sequence of preferred invention embodiments;

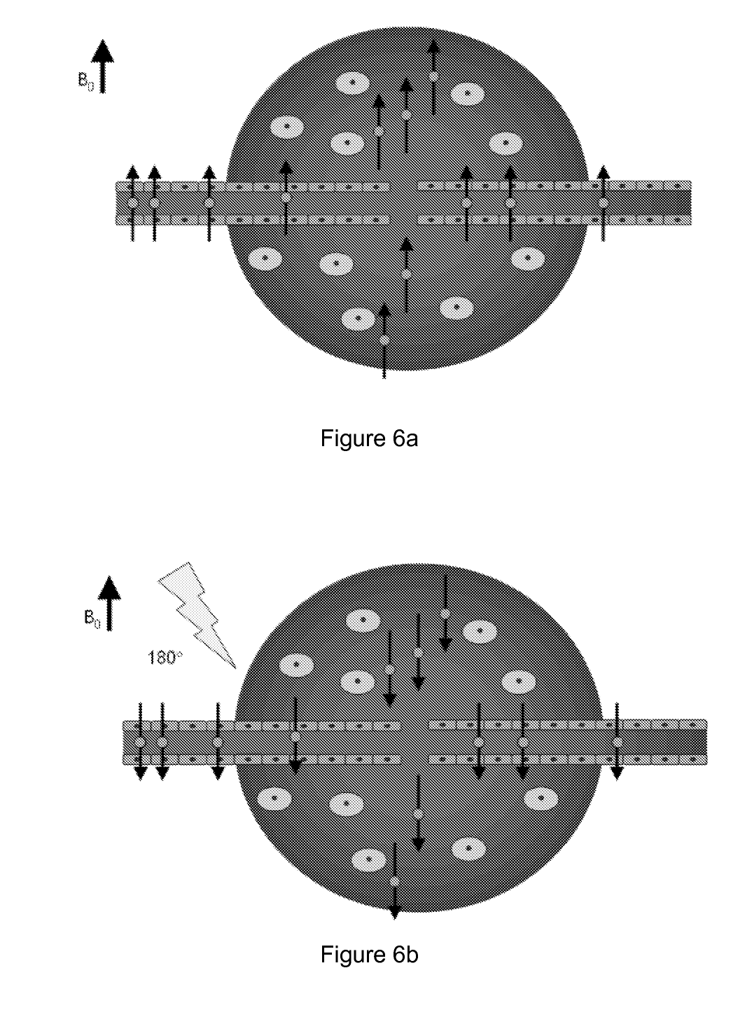

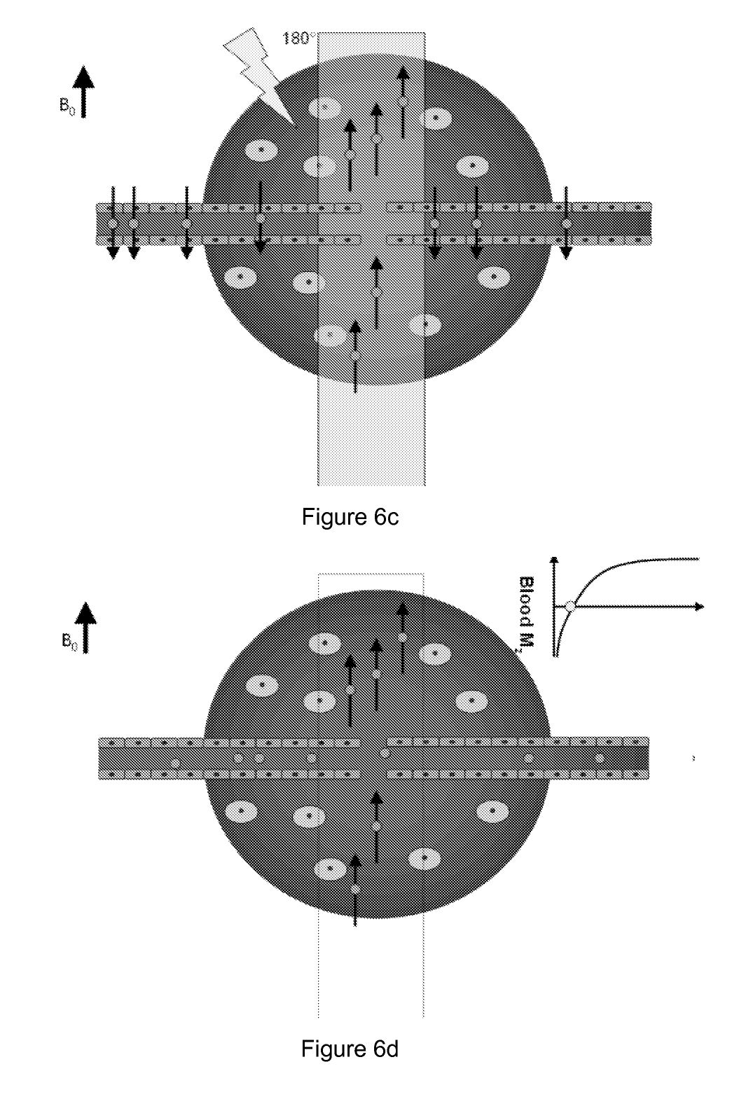

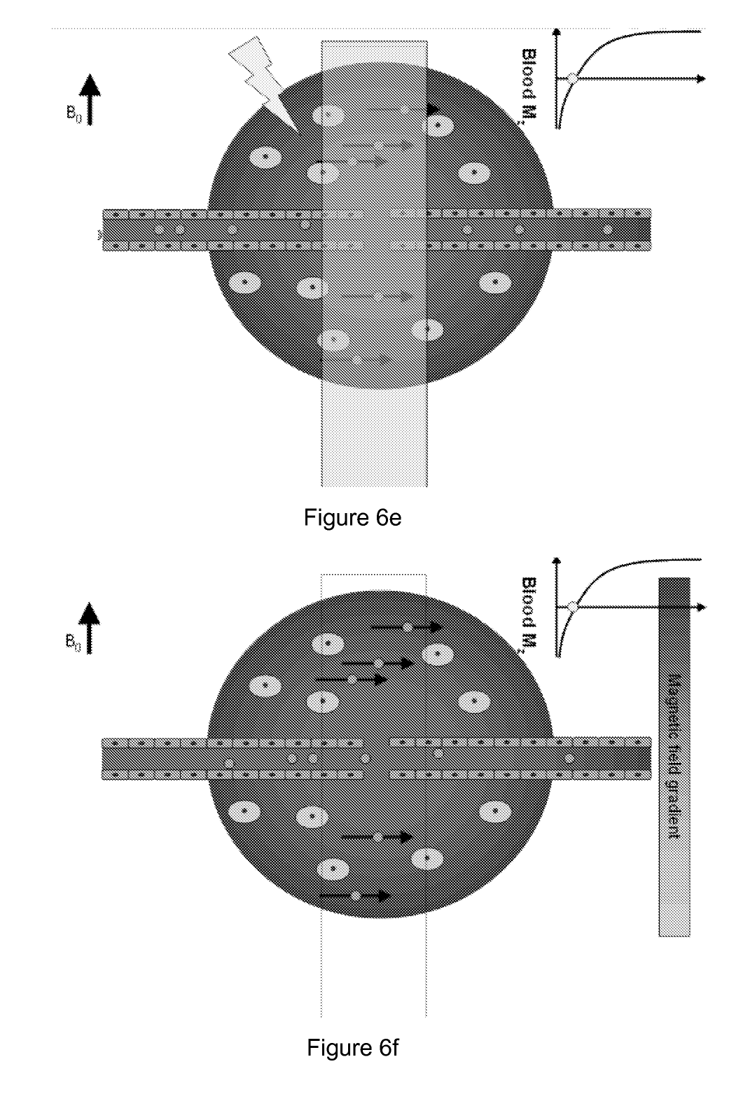

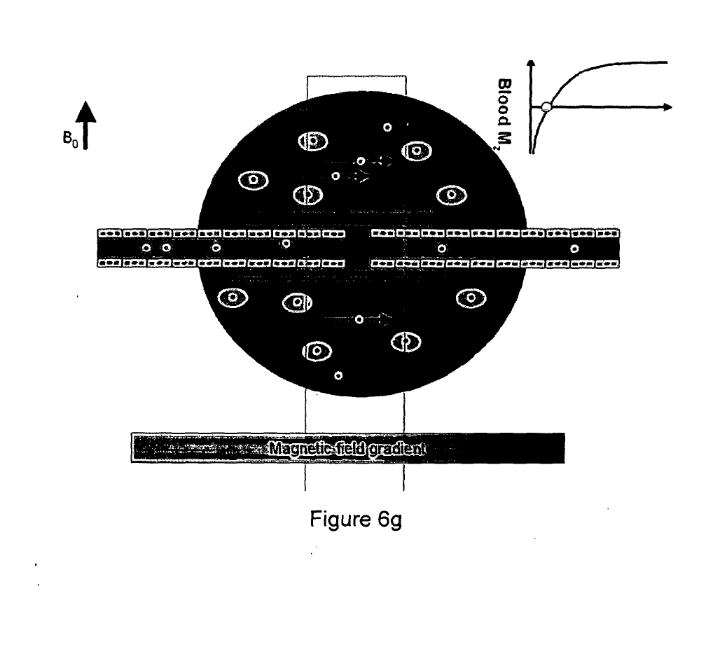

FIG. 6 is a schematic diagram to illustrate data acquisition according to invention embodiments, of which FIG. 6a shows blood flowing through tissue in a MRI scanner;

FIG. 6b shows application of a global 180° pulse;

FIG. 6c shows a slice selective 180° pulse;

FIG. 6d shows blood flowing into the slice;

FIG. 6e shows a read-out pulse administered and;

FIGS. 6f and 6g show application of velocity encoding gradients;

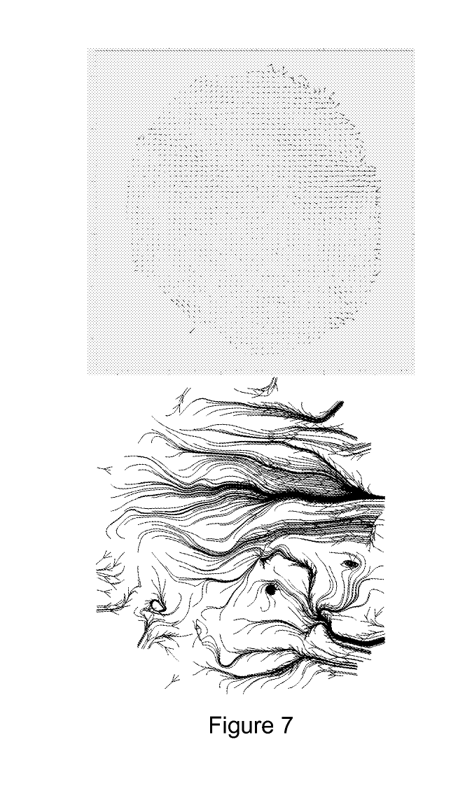

FIG. 7 shows velocity vectors produced by a preferred invention embodiment in contrast with a streamline diagram for an example tumour xenograft cross-section;

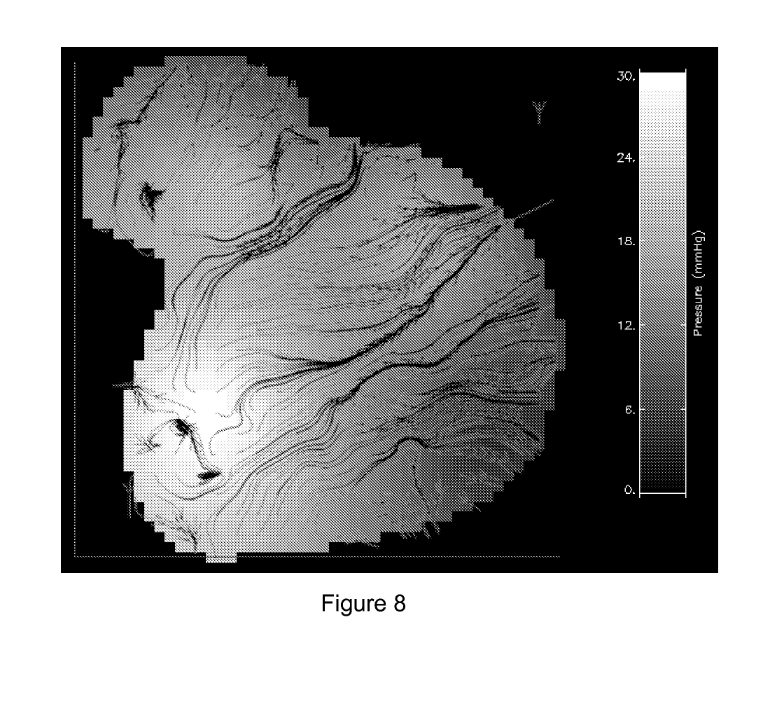

FIG. 8 shows velocity streamlines of an invention embodiment overlayed on a pressure distribution map from pressure monitor measurements;

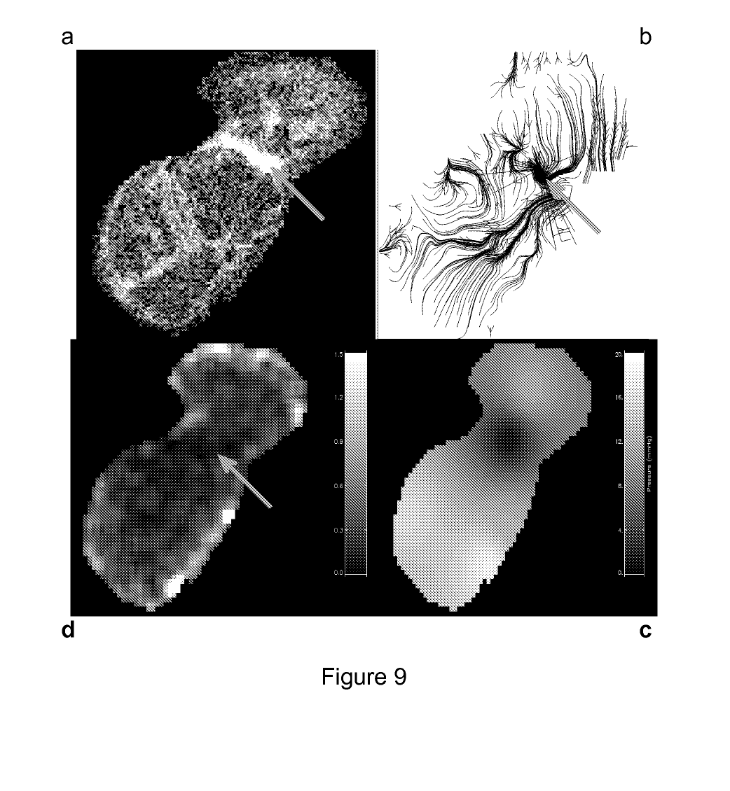

FIG. 9a shows a late Gd-DTPA enhancement image (90 minutes following start of infusion) revealing regions in which Gd-DTPA has accumulated, suggesting the presence of low pressure;

FIG. 9b shows EVAC streamlines of invention embodiments;

FIG. 9c shows a perfusion image from ASL measurement;

FIG. 9d shows pressure measurement from a hand-held monitor;

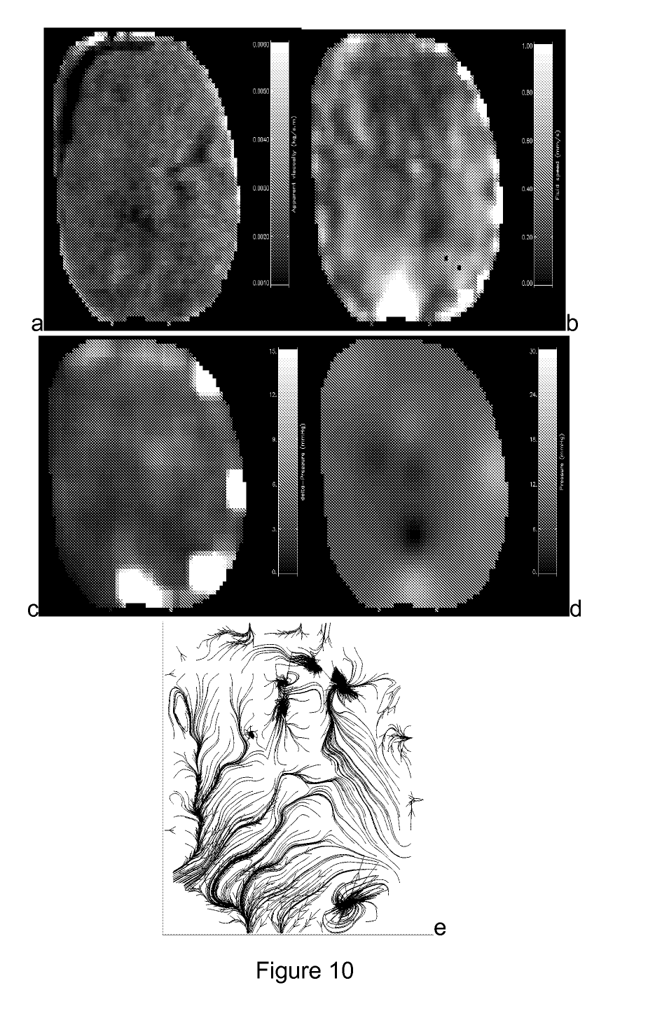

FIG. 10a shows an example map of apparent viscosity

FIG. 10b shows an example map of interstitial fluid speed;

FIG. 10c shows EVAC pressure image from a tumour cross-section according to invention embodiments;

FIG. 10d shows corresponding pressure measurement from a hand-held device; and

FIG. 10e shows an EVAC streamline image according to invention embodiments.

FIG. 3 shows a generalised embodiment of the invention. Tissue in a MRI apparatus is subjected to two MRI processes in order to investigate fluid flow/pressure within the tissue. In step 1 magnetisation of blood in blood vessels within the tissue is eliminated and in step S2, a magnetic field is applied to the regions of interest to derive fluid flow and/or pressure in step S3.

Invention embodiments relate to a novel technique referred to herein as extra-vascular convection (EVAC) MRI. It is proposed that EVAC can for example, characterise and quantify the flow of fluid through the tumour interstitium, a phenomenon of significant interest, for example in anti-cancer therapy studies. Some embodiments utilise velocity encoding to measure the convection of fluid through tissue. In order to remove the confounding effect of vascular flow, a vascular nulling preparation is applied prior to velocity encoding.

FIG. 4 is a schematic diagram of a magnetic resonance imaging apparatus (1), including radio frequency hardware, analog to digital converters and a controller in the form of a CPU (2) (or central processing unit). A console is provided for user interaction. Such appliances are well known to the person skilled in the art and further description hereof is omitted for the sake of clarity and brevity.

Conventional MRI utilises the precessional frequency of water protons when exposed to an external magnetic field. The frequency of this precession depends on the strength of this external field. Velocity encoding MRI uses magnetic field gradients to induce a spatially-dependent variation in the frequency of this precession. Protons moving with respect to this field accumulate phase in proportion to the distance travelled along this gradient.

Preferred embodiments are based on a velocity contrast sequence with a dual inversion recovery preparation as shown in FIG. 5. A global adiabatic inversion pulse (10) [4] is administered, followed immediately by a slice selective inversion (20) in order to recover the slice to equilibrium magnetisation. Following a recovery delay t_{rec} in which inverted blood flowing into the selected slice recovers to the null point (t_{rec}=In(0.5)T_{1,blood}), a gradient echo readout is applied with excitation pulse α, during which readout bipolar velocity encoding gradients are applied (for example, G=0.5 G/cm, τ=20 ms). By nulling the vascular signal with the dual inversion, phase differences measured using velocity encoding techniques known for arterial blood velocity measurement [5] should then reflect extra-vascular convection. The T₁ of blood (T_{1,blood}) was taken to be 1900 ms, as measured in the atrium of the mouse heart during a previous study, giving t_{rec}=1317 ms. Velocity encoding required two repetitions of the sequence, the second of which used bipolar gradients of opposite polarity to the first. The difference in phase between the two measurements, Δφ₁ is proportional to fluid velocity. This measurement was performed in three directions, corresponding to phase, readout and slice-select gradient orientations. (Gₚₑ, Gᵣₒ, Gₛₛ).

FIG. 6 is a schematic diagram to illustrate the EVAC acquisition of some embodiments. FIG. 6a shows blood flowing through tissue in an MRI scanner with external field B₀ aligned vertically. Water protons are shown as small paler circles in the tissue and in the blood vessel. The upward arrows denote static field (equilibrium) polarity. FIG. 6b shows how application of a global 180° pulse reorients the magnetisation of water protons antiparallel with the external field. The upward arrows denote this magnetisation. FIG. 6c shows that a slice-selective 180° pulse recovers a narrow band of proton spins (in both the blood vessel and tissue in the region of interest) back to equilibrium. FIG. 6d illustrates blood flowing into the slice which has recovered according to spin-lattice relaxation at a rate characterised by the T₁ relaxation time. At a time t_{rec} after the first inversion pulse, the blood will have no net magnetisation and this is represented in that the water protons in the blood vessel are not arrowed, whereas those in the remaining region of interest retain equilibrium. FIG. 6e at t_{rec,} a readout pulse is administered that excites only protons that were within the region of the 180° slice selective pulse (and therefore have full equilibrium magnetisation). In FIG. 6f prior to signal (echo) acquisition, velocity encoding gradients are applied in two or three spatial orientations in order to measure the velocity vector for water protons convecting within the tissue.

Relationship Between Velocity and Pressure

The velocity calculation can be used in deriving interstitial pressure. Pressure (A) is related to velocity via the Navier-Stokes equation:

$$ρ ( \frac{\partial v}{\partial t} + v \cdot ∇ v ) = - ∇ p + μ {∇}^{2} v + f . \qquad [ 1 ]$$

where v is velocity, ρ is tissue density, μ is viscosity and f takes into account any other external forces (such as gravity). The del symbol represents the spatial derivative. We assume the fluid to be non-accelerating and for other external forces to be negligible, thereby removing the influence of the first term in brackets and the final term. By performing numerical calculus of the velocity vector field acquired using EVAC, a first-order estimate of pressure gradients can be derived, although full quantification requires an estimate of the viscosity.

Diffusion MRI is an established technique for measuring the diffusion of water through tissue. It works in a similar way to velocity encoding, but utilises the change in signal magnitude (as opposed to phase) caused by a magnetic field gradient. Acquiring multiple images with increasing b-value (a measure of the diffusion weighting, given by the magnitude and spacing of bipolar field gradients) allows an estimate of the apparent diffusion coefficient (ADC) of water within the tissue.

By modelling tissue as a non-accelerating viscous fluid, we can use the diffusion of water as a probe of the hydrostatic resistance of the tissue to water convection, which we have denoted the apparent viscosity (AV). The relationship between ADC and AV is given by the Einstein-Stokes equation:

$$D - \frac{{k}_{B} T}{6 πη r} \qquad [ 2 ]$$

where D is the apparent diffusion coefficient, k_{B} is the Boltzmann constant, T is temperature, η is the apparent viscosity and r is the radius of the water molecule. Thus, by combining equations 1 and 2, we can derive an estimate of tissue fluid pressure.

In Vivo Evaluation

Six nude mice were injected subcutaneously on the lower right flank with 5×10⁶ SW1222 colorectal cancer cells. Tumours were allowed to grow to an average tumour volume of 2.1±0.5 cm³ and were scanned using a 9.4 T Varian scanner with a 39 mm birdcage coil (Rapid MR International, Columbus, Ohio). Mice were anaesthetised using isoflurane in O₂, and core body temperature was monitored and maintained at 37° using a warm air blower. Tumours were restrained using dental paste in order to remove bulk motion. A single axial slice covering the largest extent of each tumour was selected from a set of multi-slice, fast spin echo images, and was used to acquire EVAC data. The EVAC sequence included the following parameters: TR=2500 ms, TE=2.6 ms, flip angle=30°, slice thickness=1 mm, field of view=30×30 mm², matrix size=128×128. In order to evaluate the efficacy of vascular nulling, arterial spin labelling (ASL) data were acquired in the same slice and a reference, non-vascular nulled set of EVAC images (S_{noInv}) was also acquired using two global inversion pulses. The difference between this image and a normal EVAC image gave a measure of the degree of nulling; as a first order approximation, (S_{noInv}−Sᵢₙᵥ)/S_{noInv}≈v_{B}, where v_{B} is fractional blood volume.

Post-processing

The data were analysed using in-house software written in IDL (ITTVIS, Boulder, Colo.). Fluid velocity was calculated using v=Δφ/(γΔM₁) (where γ is the gyromagnetic ratio and ΔM₁ is the difference in first order velocity gradient moments). Maps of fluid velocity streamlines were calculated and visualised using the iVector tool in IDL.

Comparison with Contrast Agent Accumulation and Blood Vessel Distribution

EVAC data were acquired in 6 tumour xenograft models as described above. Following this acquisition, maps of blood perfusion were acquired using arterial spin labelling, using a flow-sensitive alternating inversion recovery (FAIR) approach [Belle]. Gadolinium-DTPA was then administered via an intra-peritoneal (i.p.) line, initially as a bolus (1 ml, 50 mM solution in saline), followed by a slow infusion for 90 minutes (2.77 μl/min). This approach reproduces that of Hassid et al. [Degani], who suggest that, once in dynamic equilibrium, Gd-DTPA should pool in regions of low tissue pressure.

Measurement of Interstitial Fluid Pressure

Following MRI scanning, interstitial fluid pressure was measured in each tumour using a hand-held pressure monitor (Stryker, Reading, UK). The monitor was modified from the standard clinical setup to include a 30 gauge needle for finer and less invasive pressure measurement. The monitor was inserted into the tumour to a depth corresponding to the MRI measurement plane and pressure measurements were recorded at between 6 and 15 sites. These pressure measurements were reconstructed into a pressure distribution using a gridding algorithm in IDL and were compared with EVAC velocity measurements.

### Results

FIG. 7 shows an example EVAC fluid velocity vector map and streamline diagram derived from a tumour cross-section. In this, and all other tumours studied, velocity profiles displayed a pattern of movement from a single or multiple sources within the tumour, towards the edge. These sources were located either towards the centre of the tumour or at the lower edge, at the interface with the abdominal muscle wall. Streamlines were directed radially from the source, towards the outermost edge of the tumour. Median fluid velocity was 0.28±0.09 mm/s.

Assessment of vascular nulling efficiency revealed a first-order estimate of V_{B}=9.69±0.05%, which is in good agreement with histological measurements of SW1222 tumour blood volume in the literature [6]. ASL estimates of tumour perfusion revealed a median of 0.28±0.16 ml/mg·min, with raised perfusion towards the periphery of the tumours.

Direct measurement of IFP using a hand-held monitor revealed a heterogeneous spatial pattern, which ranged in magnitude from 0 to 30 mmHg. An example pressure map is shown in FIG. 8, with EVAC streamlines overlaid. A clear correspondence can be observed between the source of streamlines and regions of high pressure in the tumour, and convection occurs from high to low pressure regions, as expected.

FIG. 9a shows an example late Gd-DTPA enhancement (90 minutes following the start of infusion), which shows clear regions of contrast agent accumulation that can be assumed to correspond to regions of low pressure. Comparison of the FIG. 9a map with EVAC streamlines shown in FIG. 9b reveals a correspondence between regions of contrast agent accumulation and streamline pathways. In particular, a sink point can be seen in FIG. 9b, highlighted by an arrow, which corresponds to a region of particularly raised contrast agent accumulation and also to a region of lower pressure (1 mmHg) identified with a hand-held monitor as shown in FIG. 9d. Equally, comparison of EVAC streamlines with the perfusion map shown in FIG. 9c shows a correspondence between the location of streamline source points and regions of high vascular perfusion. This suggests that EVAC can identify regions of tumour experiencing elevated pressure produced by high vascular perfusion.

FIG. 10 shows example maps of apparent viscosity (in kg/s·m), fluid speed (in mm/s), EVAC pressure (in mmHg) and pressure measured using a hand-held monitor (again in mmHg), along with an EVAC streamline map. A approximate correspondence between pressure maps can be observed, although EVAC pressure maps are lower by approximately 25%. This disparity can be partly explained by sampling discontinuities and inaccuracy in the placement of pressure transducer measurements.

### REFERENCES

[1] Boucher et al. Cancer Res., 1990; 50 (15) [2] Jain, Cancer Metastasis Review, 1990; 9(3) [3] Polacheck et al. PNAS, 2011; 108 (27) [4] Lu et al. Magn Reson Med 2005; 54 (6) [5] Pelc et al. J Magn Reson Imaging 1991; 1 (4) [6] Folarin et al., Microvasc Res. 2010; 80 (1) [7] Hassid et al., Cancer Res., 2006, 15; 66 (8) [8] Belle V, et al. J Magn Reson Imaging 1998; 8; 1249-1245 [9] Moran et al. J Magn Reson Imaging 1984; 2 (4):335-40

1. A magnetic resonance imaging (MRI) method of investigating interstitial fluid velocity in a region of interest of a tissue comprising:
substantially eliminating the magnetic resonance signal of blood flowing in one or more blood vessels in the tissue by applying a nulling preparation to the tissue; and
deriving the interstitial fluid velocity using magnetic field gradients.

2. A method according to claim 1, wherein the nulling preparation includes an inversion pulse applied to the tissue by an MRI apparatus to change the polarity of magnetism induced by a static field of the MRI apparatus.

3. A method according to claim 2, wherein the inversion pulse is global and has the effect of inverting the magnetism of the blood.

4. A method according to claim 2 or 3, wherein a further inversion pulse is then applied to a region of interest of the tissue, to reorient magnetism of the tissue in the region of interest with the original static field magnetism.

5. A method according to any of the preceding claims wherein the region of interest is a slice of a thickness between 0.3 and 4 mm, preferably between 0.5 and 3 mm.

6. A method according to any of the preceding claims, wherein velocity is derived in the region of interest after a flow time long enough to allow blood in the region of interest to be substantially replaced by inflowing blood from outside the region of interest.

7. A method according to any of the preceding claims, wherein a velocity encoding sequence of pulses is applied to derive the interstitial fluid velocity in the region of interest.

8. A method according to claim 6 or 7, wherein the velocity encoding sequence is applied at a recovery time t_{rec}=log(2)T_{1, blood,} where T_{1, blood} is the T₁ spin-lattice relaxation time of blood and t_{rec} is the time taken for the blood to recover from full inversion to the null point.

9. A method according to any of claims 6 to 8, wherein the velocity encoding sequence comprises, in each of one or more directions, two applications of a sequence, the second using gradients of a different polarity, and the difference in phase between echo measurements after the two applications being proportional to the velocity.

10. A method according to claim 9, wherein the gradients are bipolar gradients of opposite polarity, and wherein the encoding sequence is applied in one, two or preferably three orthogonal spatial orientations.

11. A method according to claim 9 or 10, wherein the velocity is calculated by taking into account the gyromagnetic ratio of water protons and the difference in first order velocity encoding gradient moments for the two measurements.

12. A method according to any of the preceding claims, further comprising displaying an image of interstitial fluid velocity.

13. A method according to any of the preceding claims, further including measuring the T₁ relaxation time of blood in situ as a first process.

14. A method of predicting drug delivery and/or response to therapy of a tissue including investigating fluid flow in a tissue as claimed in any of claims 1 to 13.

15. A method according to claim 14, wherein the tissue is tumour tissue or stroke-damaged brain tissue or tissue affected by compartment syndrome.

16. A method of measuring pressure of tumour interstitium comprising using the tissue investigation method of any of claims 1-15, and then using interstitial fluid velocity to calculate interstitial fluid pressure.

17. A pressure measurement method according to claim 16, wherein the pressure is calculated by applying diffusion MRI sequences to investigate diffusion within the tissue in conjunction with use of the Navier-Stokes and Einstein-Stokes equations to derive pressure.

18. A magnetic resonance imaging (MRI) apparatus for investigating interstitial fluid velocity in a region of interest of a tissue of a subject, the MRI apparatus including a controller operable to control the applied magnetic fields within the MRI apparatus to:
substantially eliminate the magnetic resonance signal of blood flowing in one or more blood vessels in the tissue by application of a nulling preparation; and
apply a velocity encoding sequence allowing derivation of the interstitial fluid velocity.

19. A computer which is programmed to accept data from a magnetic resonance imaging (MRI) apparatus for investigating interstitial fluid velocity in a tissue of a subject, the MRI apparatus including a controller operable to:
substantially eliminate magnetic resonance of blood in the tissue by application of a nulling sequence; and
apply a velocity encoding sequence allowing derivation of the interstitial fluid velocity; wherein
the data includes echo data representing signals emitted from the tissue in response to the velocity encoding sequence and the computer is also programmed to derive interstitial fluid velocity by processing the echo data.

20. A computer according to claim 17, further comprising a user interface including an output device to allow the user to view an image of interstitial fluid velocity and an input device to allow the user to control the MRI apparatus programming.

21. An MRI system including a magnetic resonance imaging (MRI) apparatus according to claim 18 and a computer according to claim 19 or 20.

22. A computer program which when executed by an MRI system causes it to carry out the method of any of claims 1 to 17.

23. A tangible, non-transitory computer-readable medium storing a computer program according to claim 22.

24. A method, MRI apparatus, computer or MRI system substantially according to one of the embodiments shown in the figures and/or detailed in the description.
